(19) United States
(12) Patent Application Publication
(10) Pub. No.: US 2021/0393844 A1
(43) Pub. Date: Dec. 23, 2021

(54) VIRUS PROTECTION SHIELD

(71) Applicant: LEE ARTHUR DAWSON, MANGO HILL (AU)

(72) Inventor: LEE ARTHUR DAWSON, MANGO HILL (AU)

(21) Appl. No.: 17/353,056

(22) Filed: Jun. 21, 2021

Related U.S. Application Data
(60) Provisional application No. 63/042,586, filed on Jun. 23, 2020.

(51) Int. Cl. A61L 9/20

(52) U.S. Cl. CPC A61L 9/20; A61L 2209/15; A61L 2209/12

(57) ABSTRACT

The inventive concept is a virus protection shield for a user that includes a rechargeable LED ultraviolet light emitter assembly coupled to a protective mask or a frame superposing the user's mouth, the LED ultraviolet light emitter portion disposed on a distal face of the LED ultraviolet light emitter assembly and designed to emit ultraviolet light substantially outward from the user. An actuator assembly is designed to render the LED ultraviolet light emitter portion into an emitting or non-emitting state.

### CLAIM OF PRIORITY

This application claims priority to and the benefit of U.S. Provisional application with Ser. No. 63/042,586, filed on Jun. 23, 2020, entitled VIRUS PROTECTION SHIELD, which is hereby incorporated by reference in its entirety.

### FIELD OF THE INVENTION

The inventive concept relates generally to a virus protection shield.

### BACKGROUND

Currently, there are a number of solutions for blocking viruses and bacteria from infecting a person. One of these solutions is to wear a mask, but this solution fails to meet the needs of the market because masks may let viruses in through exposed sides or through the fabric. Another solution attempts to cover the mouth, but this solution is similarly unable to meet the needs of the market because it does not protect a person if other people are carrying a virus. Still another solution seeks to utilize disinfectant, but this solution also fails to meet market needs because disinfectants do not protect the face. Therefore, there currently exists a need in the market for a virus protection shield that blocks viruses and bacteria.

### SUMMARY OF THE INVENTION

The inventive concept is a virus protection shield for a user. In one embodiment, a headset assembly has a flexible frame assembly coupled by a proximal end of the frame assembly to the headset assembly, a distal end of the frame assembly designed to be substantially superposing the user's mouth. A rechargeable LED ultraviolet light emitter assembly is coupled to the distal end of the frame assembly, the LED ultraviolet light emitter portion disposed on a distal face of the LED ultraviolet light emitter assembly and designed to emit ultraviolet light substantially outward from the user, the substantially outward emissions creating a protective shield substantially over the face. An actuator assembly is coupled to the flexible frame assembly designed to render the LED ultraviolet light emitter portion into an emitting or non-emitting state.

One embodiment of the virus protection shield for a user has a substantially planar magnet assembly disposed to be on a proximal surface of a protective mask assembly such as a surgical mask or other mask used to shield against dust, viruses, and bacteria. A rechargeable LED ultraviolet light emitter assembly has a proximal face and a distal face, the LED ultraviolet light emitter portion disposed on the distal face of the LED ultraviolet light emitter assembly. A substantially planar and ferrous metal assembly is coupled to the proximal face of the LED ultraviolet light emitter assembly, the LED ultraviolet light emitter assembly designed to be coupled to a distal surface of the protective mask assembly by electromagnetic waves passing through the protective mask assembly from the substantially planar magnet assembly. The LED ultraviolet light emitter assembly, when so coupled, is designed to emit ultraviolet light substantially outward from the user. An actuator assembly is disposed on the proximal face of the LED ultraviolet light emitter assembly designed to render the LED ultraviolet light emitter portion into an emitting or non-emitting state.

In one embodiment of the virus protection shield for a user, the ultraviolet light is emitted at or within the range of 207-222 nm, often known as Far-UVC.

In one embodiment of the virus protection shield, the inventive concept has a charging port and is rechargeable and reusable. The inventive concept may have a rubber headset.

In one embodiment, the inventive concept may include detachable magnets and house at least one pcb rechargeable battery and at least one Far UVC 222 nm light source that will fit onto most masks and is designed with hook and loop attachments to most other surfaces. The inventive concept may be used to help personal wearers and or placed onto cots cribs inside vehicles to give added protection to the user. The inventive concept may adapt to an ear over carrier to be used wear wearing masks may be inconvenient for the user to wear a mask and or it can be worn with a mask. The magnetic light source may be attached back to back with another magnetic light to allow one light to shine forward and the second to shine backwards thus giving 2-way protection to the user. Devices may be rechargeable with 3-volt coin cell batteries and has a warning light for a low battery status the ear over attachment that may be made of flexible wire and silicon for wearer comfort. The inventive concept may be designed and used for multiple applications, such as public transport, air travel, ships, taxis, passenger vehicles, or inside ATMs to disinfect cash. The inventive concept may be attached to baby prams and cots.

It would be advantageous to have a virus protection shield that is portable. Furthermore, it would be advantageous to have a virus protection shield that is reusable. Still further, it would be advantageous to have a virus protection shield that is rechargeable. The Far-UVC light creates a shield around the face area against viruses and bacteria.

The inventive concept advantageously fills the aforementioned deficiencies by providing a virus protection shield, which provides a way to kill viruses using Far-UVC light.

Far-UV light has a shorter wavelength and higher photon energy than UV-C, UV-B, and UV-A, making, its effect on microorganisms different from UV disinfection lamps that are most commonly used. Far-UV light is able to cause physical destruction of viral, bacterial, and fungal cells in a matter of seconds, much faster than UV-C lamps. Continuous low doses of far-ultraviolet C (far-UVC) light can kill airborne flu viruses without harming human tissues. But because viruses and bacteria are much smaller than human cells, far-UVC light can reach their DNA and kill them.

The inventive concept may be used as a far-UVC light emitting magnet.

Among other things, it is an advantage of the virus protection shield to provide a virus protection shield that does not suffer from problems or deficiencies associated with prior solutions.

It is still further an advantage of the virus protection shield to be reusable.

Still further, the virus protection shield has an over-the-ear and fabric mask version of the virus protection shield.

The inventive concept now will be described more fully hereinafter with reference to the accompanying drawings, which are intended to be read in conjunction with both this summary, the detailed description, and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete, and will fully convey the full scope of the inventive concept to those skilled in the art.

### BRIEF DESCRIPTION OF THE DRAWINGS

FIG. 1 illustrates a front view of the over the ear version of the virus protection shield.

FIG. 2 illustrates a back or rear view of the over the ear version of the virus protection shield.

FIG. 3 illustrates a right side view of the over the ear version of the virus protection shield.

FIG. 4 illustrates a left side view of the over the ear version of the virus protection shield.

FIG. 5 illustrates the top view of the over the ear version of the virus protection shield.

FIG. 6 illustrates the bottom view of the over the ear version of the virus protection shield.

FIG. 7 illustrates the perspective view of the over the ear version of the virus protection shield.

FIG. 8 illustrates the front view of the mask version of the virus protection shield.

FIG. 9 illustrates the back view of the mask version of the virus protection shield.

FIG. 10 illustrates the top view of the mask version of the virus protection shield.

FIG. 11 illustrates the bottom view of the mask version of the virus protection shield.

FIG. 12 illustrates the right side view of the mask version of the virus protection shield.

FIG. 13 illustrates the left side view of the mask version of the virus protection shield.

FIG. 14 illustrates the perspective view of the mask version of the virus protection shield.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

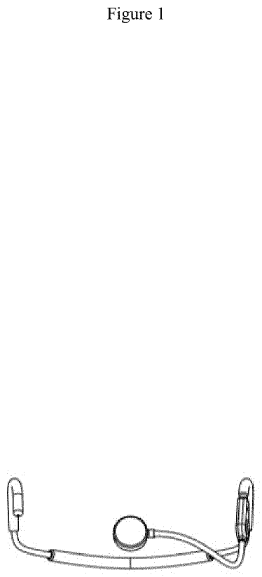

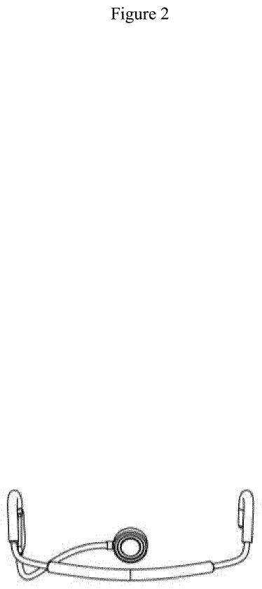

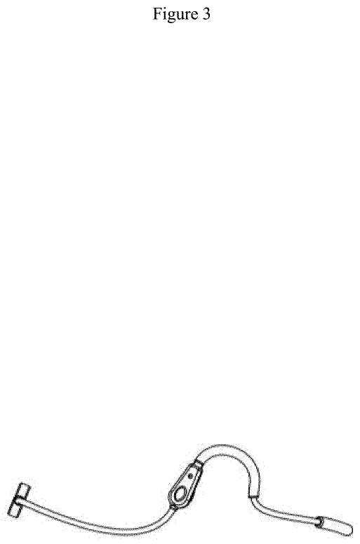

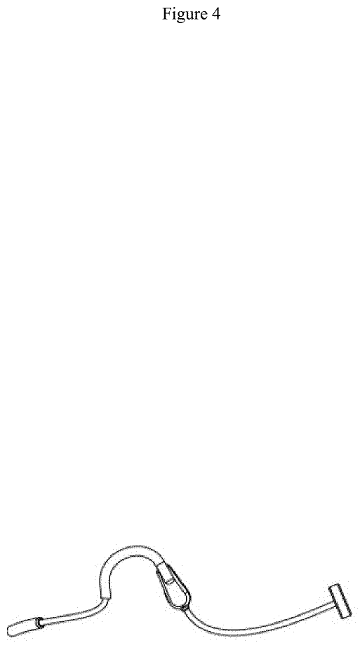

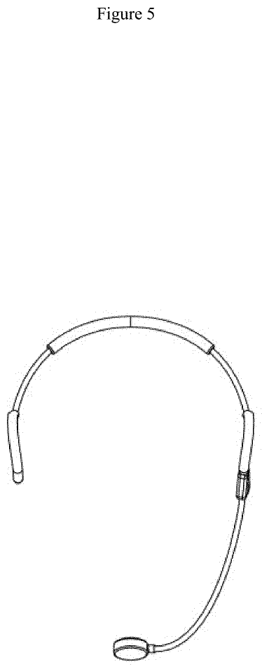

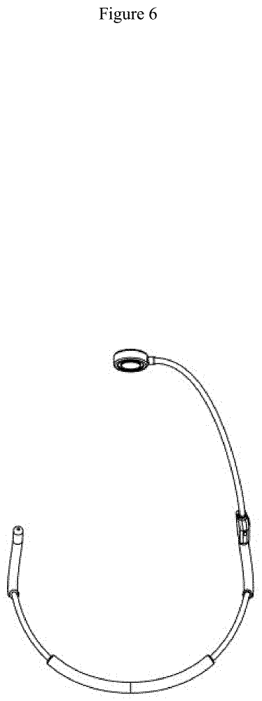

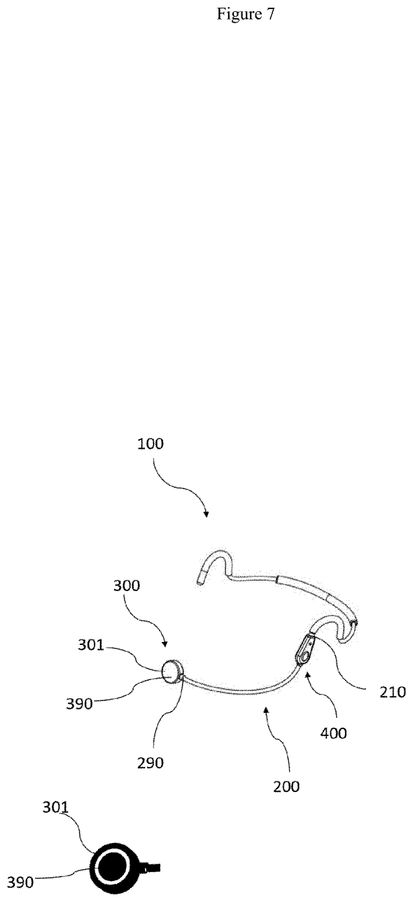

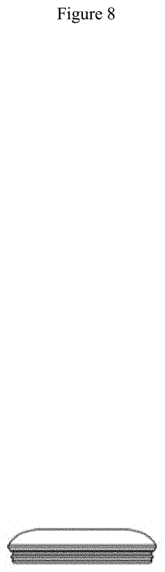

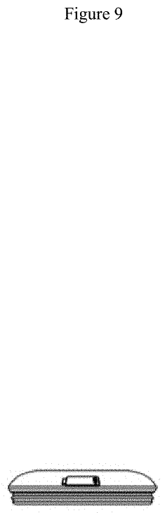

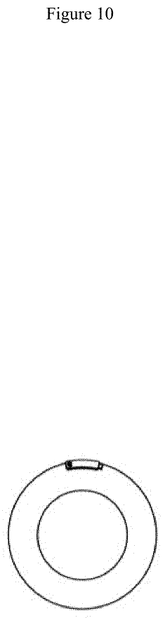

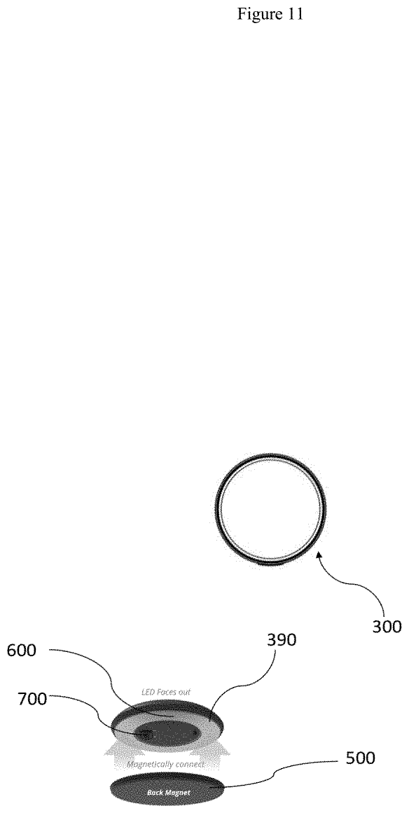

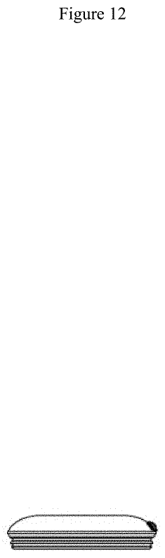

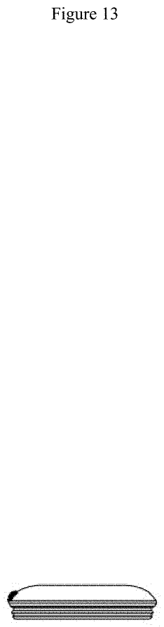

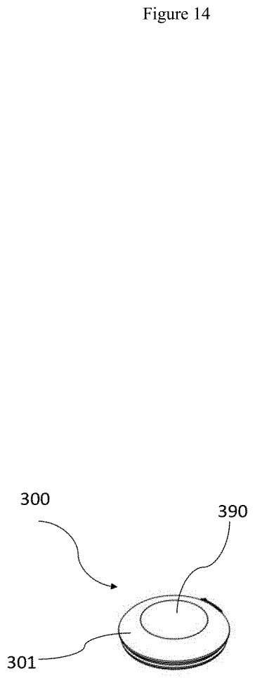

Referring to the figures, FIGS. 1-14 illustrate one embodiment that is a headset assembly 100 that has a flexible frame assembly 200 coupled by a proximal end of the frame assembly 210 to the headset assembly 100, a distal end of the frame assembly 290 designed to be substantially superposing the user's mouth. A rechargeable LED ultraviolet light emitter assembly 300 is coupled to the distal end of the frame assembly 290, the LED ultraviolet light emitter portion 301 disposed on a distal face 390 of the LED ultraviolet light emitter assembly 300 and designed to emit ultraviolet light substantially outward from the user. An actuator assembly 400 is coupled to the flexible frame assembly 200 designed to render the LED ultraviolet light emitter portion 301 into an emitting or non-emitting state.

In one embodiment of the virus protection shield for a user, a substantially planar magnet assembly 500 is disposed to be on a proximal surface of a protective mask assembly such as a surgical mask. A rechargeable LED ultraviolet light emitter assembly 300 has a proximal face 310 and a distal face 390, the LED ultraviolet light emitter portion 301 disposed on the distal face 390 of the LED ultraviolet light emitter assembly 300. A substantially planar and ferrous metal assembly 600 is coupled to the proximal face 310 of the LED ultraviolet light emitter assembly 300, the LED ultraviolet light emitter assembly 300 designed to be coupled to a distal surface of the protective mask assembly by electromagnetic waves passing through the protective mask assembly from the substantially planar magnet assembly 500. The LED ultraviolet light emitter assembly 300, when so coupled, is designed to emit ultraviolet light substantially outward from the user. An actuator assembly 700 is disposed on the proximal face 310 of the LED ultraviolet light emitter assembly 300 designed to render the LED ultraviolet light emitter portion 301 into an emitting or non-emitting state.

The following patents are incorporated by reference in their entireties: U.S. Pat. Nos. 5,165,395, 6,752,627, 10,517,976, and 20110162155A1. Also referenced in use is the the US Far UVC 222n.

While the inventive concept has been described above in terms of specific embodiments, it is to be understood that the inventive concept is not limited to these disclosed embodiments. Upon reading the teachings of this disclosure many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A virus protection shield for a user comprising:
a headset assembly;
a flexible frame assembly coupled by a proximal end of the frame assembly to the headset assembly, a distal end of the frame assembly adapted to be substantially disposing over or superposing the user's mouth;
a rechargeable LED ultraviolet light emitter assembly coupled to the distal end of the frame assembly, the LED ultraviolet light emitter portion disposed on a distal face of the LED ultraviolet light emitter assembly and adapted to emit ultraviolet light substantially outward from the user; and
an actuator assembly coupled to the flexible frame assembly adapted to render the LED ultraviolet light emitter portion into an emitting or non-emitting state.

2. A virus protection shield for a user comprising:
a substantially planar magnet assembly disposed to be on a proximal surface of a protective mask assembly;
a rechargeable LED ultraviolet light emitter assembly with a proximal face and a distal face, the LED ultraviolet light emitter portion disposed on the distal face of the LED ultraviolet light emitter assembly;
a substantially planar and ferrous metal assembly coupled to the proximal face of the LED ultraviolet light emitter assembly, the LED ultraviolet light emitter assembly adapted to be coupled to a distal surface of the protective mask assembly by electromagnetic waves passing through the protective mask assembly from the substantially planar magnet assembly, the LED ultraviolet light emitter assembly, when so coupled, adapted to emit ultraviolet light substantially outward from the user; and
an actuator assembly disposed on the proximal face of the LED ultraviolet light emitter assembly adapted to render the LED ultraviolet light emitter portion into an emitting or non-emitting state.

3. The virus protection shield for a user of claim 1 wherein the ultraviolet light is emitted at or within the range of 207-222 nm.

4. The virus protection shield for a user of claim 2 wherein the ultraviolet light is emitted at or within the range of 207-222 nm.
